(19) United States
(12) Patent Application Publication
(10) Pub. No.: US 2019/0169561 A1
(43) Pub. Date: Jun. 6, 2019

(54) IN-SITU CELL RETENTION PERFUSION BIOREACTORS

(71) Applicant: PBS Biotech, Inc., Camarillo, CA (US)

(72) Inventors: Chanyong Brian Lee, Thousand Oaks, CA (US); Yasunori Hashimura, Woodland Hills, CA (US); Gary Evans, Ventura, CA (US); Sunghoon Jung, Camarillo, CA (US)

(21) Appl. No.: 16/210,238

(22) Filed: Dec. 5, 2018

Related U.S. Application Data
(60) Provisional application No. 62/609,142, filed on Dec. 21, 2017.
(60) Provisional application No. 62/595,464, filed on Dec. 6, 2017.

(51) Int. Cl. C12M 1/34; C12M 1/00; C12M 3/04; C12N 5/07; C12M 1/02; B01L 3/00

(52) U.S. Cl. CPC C12M 41/16; C12M 29/04; C12M 41/32; C12M 29/10; C12M 3/04; C12N 5/06; B01L 2300/0861; C12M 1/02; C12M 41/14; B01L 3/502; B01L 2200/0647; B01L 2300/0681; B01L 2300/0848; C12M 1/005

(57) ABSTRACT

The bioreactor may include a single-use, rigid-sided bioreactor vessel containing a fluid to be mixed and a vertical mixing wheel. A perfusion dip tube with a screen filter incorporated on the end is secured to the vessel from the top lid and partially submerged in the fluid, preferably into close proximity with an outer circumference of the vertical mixing wheel. The screen filter of appropriate mesh size allows spent cell culture medium to be withdrawn from the bioreactor vessel while retaining cell aggregates or microcarriers on which cells are attached and growing in the vessel. Alternating flow of fluid out from and into the dip tube enables removal of spent medium and unclogging of the dip tube filter.

### RELATED APPLICATIONS

The present application claims priority to U.S. Provisional Application Nos. 62/609,142 filed Dec. 21, 2017, and 62/595,464 filed Dec. 6, 2017, the disclosures of which are expressly incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

A bioreactor system and methods of use therefor.

### BACKGROUND

Efforts of biopharmaceutical companies to discover new biological drugs have increased exponentially during the past two decades. Bioreactors have been used for cultivation of animal and plant cells as well as microbial organisms for production of various biological or chemical products in the pharmaceutical, biotechnology, and beverage industries. Most biological drugs are produced by cell culture or microbial fermentation processes which require sterile bioreactors and an aseptic culture environment. A production bioreactor provides various nutrients that are required to support maintenance and growth of biological agents of interest and also uses a stirring mechanism to promote homogenous mixing of culture medium.

Well-established cell lines, such as Chinese Hamster Ovarian (CHO) cells, have been adapted from adherent culture over time to grow in single-cell suspension.

For the cell therapy market, however, the desired products are often human primary cells such as embryonic stem cells, adult stem cells (e.g., mesenchymal stem/stromal cells), and induced pluripotent stem cells that are newly derived from human donors, shear-sensitive, and anchorage dependent in nature (i.e., they will grow in aggregates or on scaffolds such as tissue-culture plates or microcarriers in a bioreactor containing cell culture medium). During cell culture processes, spent medium often needs to be exchanged with fresh medium to ensure that key nutrients are not depleted and toxic metabolic by-products such as lactate and ammonia do not accumulate to a level that inhibits cell growth. Cell culture medium exchanges for primary cells are typically performed in discrete mode of removing spent medium first and then replacing with fresh medium; typically, a majority of spent liquid media is removed before being replaced with fresh media. However, this approach may lead to abrupt changes in key process parameters such as pH, dissolved, oxygen, and temperature, or may also damage primary cells or permit them to clump together as they settle to the bottom of a bioreactor. In contrast, perfusion culture mode removes spent medium while simultaneously adding fresh medium in a continuous or semi-continuous manner without disturbing nutrient and mixing parameters. Thus cells are retained and mixed homogenously without interruption in the bioreactor, and perfusion represents a better medium exchange method, especially for cell therapy applications.

Despite a proliferation of single-use bioreactor designs for culturing primary cells, the existing options for perfusion culture modes are based on external devices which can potentially cause shear damage during external circulation of culture media. Thus there is a need for an improved perfusion technique that can be integrated into the bioreactor without an external circulation of the culture media.

### SUMMARY OF THE INVENTION

The present application discloses a perfusion configuration for bioreactors that allows users to culture cells by providing fresh culture media in a continuous or semi-continuous manner in lieu of medium exchanges. The techniques are preferably though not exclusively used for culturing primary cells. A single-use, rigid-sided bioreactor vessel is positioned within a vertical mixing wheel bioreactor system. A perfusion dip tube with a screen filter incorporated on the end would be secured to the vessel from the top lid and partially submerged in the medium, preferably into close proximity with an outer circumference of a vertical mixing wheel. The screen filter of appropriate mesh size would allow spent cell culture medium to be withdrawn from the bioreactor vessel while retaining cell aggregates or microcarriers on which cells are attached and growing in the vessel.

Preferably, the end of the dip tube with the screen filter is positioned close to the outer circumference of the vertical mixing wheel so that tangential fluid flow generated from the wheel rotation can help dislodge cells that could otherwise clog the filter. Furnishing a bioreactor with a dip tube in this configuration offers the ability to remove spent culture medium from the bioreactor at an automated liquid flow rate to mitigate the risk of cells clogging the filter. As spent culture medium is removed from the bioreactor through the dip tube, fresh culture medium is added at the same rate through a different line on the bioreactor to maintain a desired medium volume within the vessel in a continuous or semi-continuous manner.

In another embodiment of the invention, the outer end of the dip tube also contains a branch point that splits into two lines—the first line for removing spent culture medium from the bioreactor vessel and the second line for introducing a known volume of sterile liquid or gas into the bioreactor vessel to clear the screen filter of any cells that might collect on its surface. Sterile liquid that is introduced in the second line could be fresh culture medium that is added to replace the spent culture medium that has been withdrawn. The flow rate and duration of liquid or gas addition would be optimized depending on the type of cells, the size of the cell aggregates, and the concentration of cell aggregates in the bioreactor vessel to ensure cells are adequately dislodged from the screen filter without imparting shear damage on the cells. In this embodiment, the perfusion dip tube would operate in a semi-continuous manner to allow some duration of time for spent medium withdrawal, followed by some duration of time for cell dislodging from the screen filter on the dip tube.

### DESCRIPTION OF THE DRAWINGS

FIG. 1 illustrates a single-use vessel positioned within a bioreactor system and showing an exemplary perfusion dip tube with a screen filter;

FIG. 2 is a perspective view of the single-use vessel from above with an upper lid removed and showing the position of the perfusion dip tube relative to a vertical mixing wheel therein;

FIGS. 3A-3C are orthogonal views of the single-use vessel, vertical mixing wheel and dip tube shown in FIG. 2; and

FIG. 4 is a side view of the single-use vessel, vertical mixing wheel and dip tube with an alternative branched inlet/outlet configuration.

### DETAILED DESCRIPTION

The present application provides a perfusion dip tube for a bioreactor system which offers all of the benefits of a harvest port, while allowing cell-free liquid to be collected and replaced multiple times throughout a run, which is advantageous when dealing with culturing of primary cells.

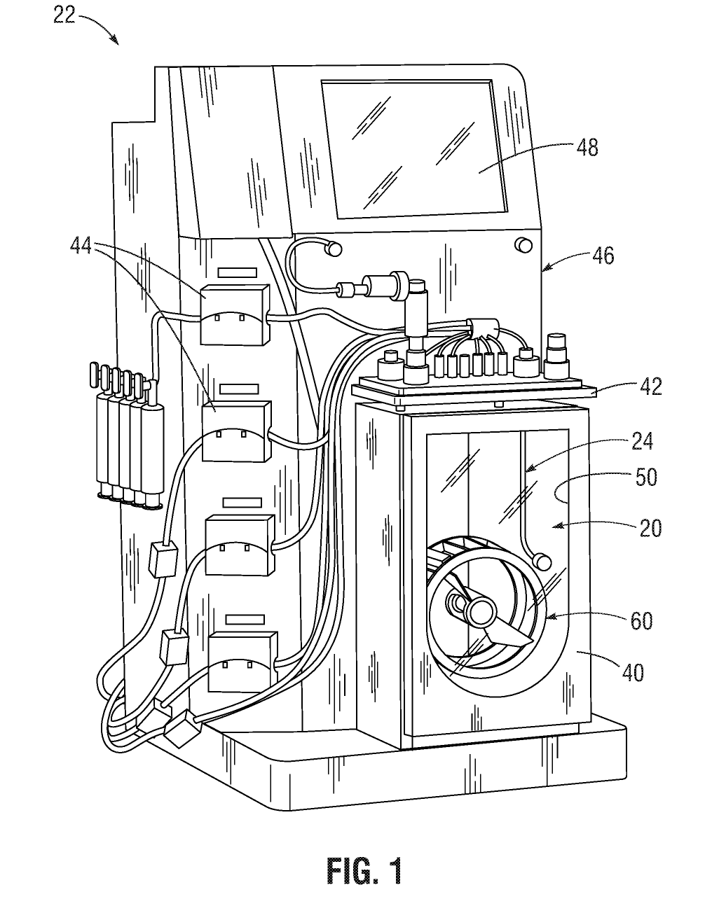

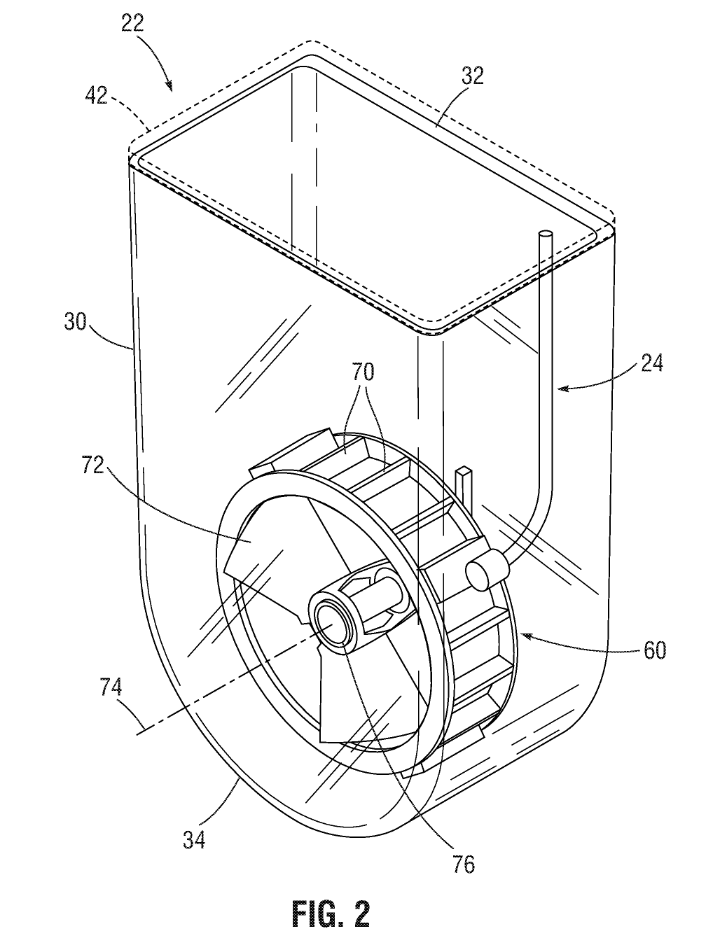

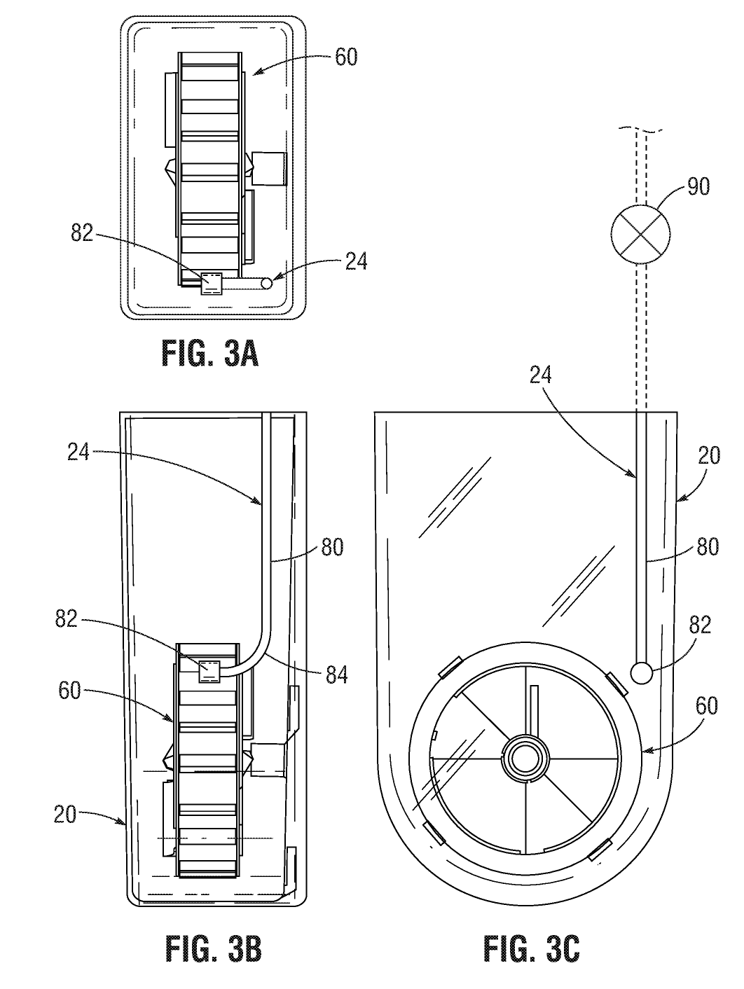

FIG. 1 illustrates a single-use vessel 20 positioned within a bioreactor system 22 and showing an exemplary perfusion dip tube 24 in the vessel. The vessel 20 is shown isolated in FIGS. 2-3 and comprises a generally rectangular, rigid plastic-walled container 30 having a top edge 32, a generally rectangular upper section and a closed semi-cylindrical bottom end 34.

With reference again to FIG. 1, the vessel 20 is placed within a closely surrounding rectangular box-shaped frame 40 and an upper lid 42 secured over the top edge 32. The upper lid 42 provides access ports for a plurality of supply conduits and measurement instruments. Some of the supply conduits pass through one of a plurality of peristaltic pumps 44 mounted to a larger control housing 46. A display screen 48 at the top of the control housing 46 provides a user interface and feedback indicators.

FIG. 1 also shows an open front window 50 in the frame 40 through which the clear-walled vessel 20 and its contents can be seen. In a preferred embodiment, a vertical-mixing wheel 60 positioned within the vessel 20 rotates about a horizontal axis and stirs the culture medium within the vessel. The mixing wheel 60 is positioned lower down in the vessel 20 so as to be substantially evenly spaced around its circumference from the semi-cylindrical bottom end 34.

With reference to FIGS. 2 and 3A-3C, the L-shaped dip tube 24 is shown extending down from the upper lid 42 (shown in phantom in FIG. 2) into proximity with the mixing wheel 60. The dip tube 24 may be provided as a built-in feature of the upper lid 42 or may be provided separately, but in either case is rigidly supported by the upper lid 42. For instance, the dip tube 24 may pass through a vertical aperture (not shown) in the upper lid 42 and be secured therein by a clamp or other such fitting. Alternatively, the dip tube 24 may be fixed within a vertical aperture (not shown) in the upper lid 42 such as with adhesive or thermal bonding or the like.

Preferably, the vertical mixing wheel 60 features a series of angled- or radially-oriented vanes 70 on its exterior for stirring the solution within the container 22, and also may include centrally positioned vanes 72 that are curved to produce axial flow. The wheel 60 rotates about a horizontal axis 74 on hubs 76 secured to the front and/or back walls of the container 22. In a preferred embodiment, the control housing 46 includes a drive system including rotating magnetic drive elements (not shown). Corresponding driven elements such as magnets or ferromagnetic material mounted around the wheel 60 allow coupling of the drive system to enable rotation of the wheel from outside the container 22, thus eliminating seals and the like which might contaminate the solution within the container. An exemplary magnetic drive system is seen in U.S. Patent Publication No. 2015/0175951, which is expressly incorporated herein. Alternatively, an air bubble drive may be utilized, as disclosed in U.S. Pat. No. 7,628,528, also expressly incorporated herein.

In a preferred embodiment, the volumetric capacity of the container 22 is between 0.5-3.0 L, although the system can be scaled up for larger capacities.

As seen best in FIG. 3B, the dip tube 24 has an elongated vertical section 80 extending down from the upper lid 42 that transitions at a gently curved approximately 90° elbow section 84 to a horizontally-oriented section terminating in an input/output port 82. The input/output port 82 preferably comprises a cylindrical filter over an open lower end of the dip tube 24. The axis of the horizontally-oriented input/output port 82 and cylindrical filter is desirably parallel to the rotational axis 74 of the wheel 60.

An upper end of the dip tube 24 connects with a liquid supply and removal system through a bi-directional pump 90. The pump 90 may be one of the peristaltic pumps 44 shown in FIG. 1, or a standard impeller-type of pump.

The position and orientation of the input/output port 82 relative to the vertical mixing wheel 60 facilitates removal and injection of culture media. That is, the cylindrical filter at input/output port 82 is oriented with its axis parallel to the wheel 60 axis and positioned closely adjacent to the outer circumference thereof. Preferably, the cylindrical filter at input/output port 82 is located closely adjacent to the rotating wheel 60 above the rotational axis 74 (see FIG. 2), and more preferably at about a 45° angle extending from the horizontal axis 74 above a horizontal plane through the axis 74. The tangential flow generated by the rotating wheel washes any cells or microcarriers that might otherwise aggregate on the surface of the filter and hinder or block flow therethrough. In a preferred embodiment, the input/output port 82 is spaced a distance of between about 1-3 inches away from the outer circumference of the rotating mixing wheel 60. Further, the input/output port 82 is positioned on a vertical mid-plane of the wheel 60 as seen in FIG. 3B to even out flow around the filter and help wash away any blockages.

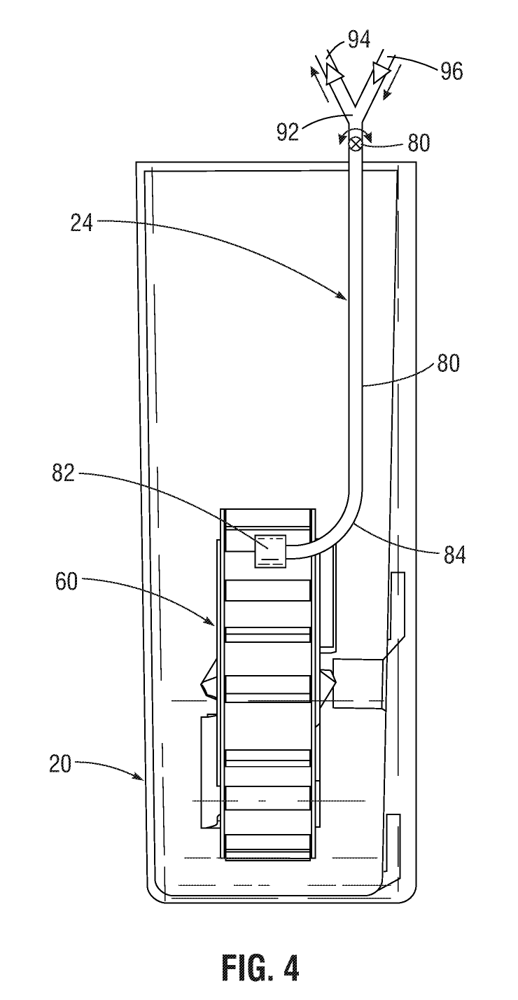

FIG. 4 is a side view of the single-use vessel 20, vertical mixing wheel 60 and dip tube 24 with an alternative inlet/outlet configuration. Specifically, an upper end of the dip tube 24 contains a branch point 92 that splits into two lines—a first line 94 for removing spent culture medium from the bioreactor vessel and a second line 96 for introducing a known volume of sterile liquid or gas into the bioreactor vessel to clear the screen filter of any cells that might collect on its surface. A valve 98 at the branch point 92 enables conversion between the two lines, and respective flow pumps (not shown) are provided for each line. Sterile liquid that is introduced in the second line 96 could be fresh culture medium that is added to replace the spent culture medium that has been withdrawn. The perfusion dip tube 24 would operate in a semi-continuous manner to allow some duration of time for spent medium withdrawal, followed by some duration of time for cell dislodging from the screen filter on the dip tube.

The filter at the input/output port 82 is desirably made of a porous material that is gamma-sterilizable, so that the entire vessel 20 may be sterilized prior to a culture batch run. For instance, the filter may be sintered polyethylene mesh, polyvinylidene fluoride (PVDF) mesh, or even stainless steel mesh.

The bioreactor vessel and system disclosed herein permits different modes of use such as continuous or semi-continuous perfusion, while capturing the desired cells grown on microcarriers or as aggregates. An expected size range of micro carriers or aggregates is approximately 50-500 micron in diameter.

Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A perfusion bioreactor, comprising:
a. a vessel having an upper lid and sized to contain a volume of fluid or medium;
b. a vertically-oriented mixing wheel positioned within the vessel and having a horizontally-oriented axle supported within the vessel about which the mixing wheel rotates, the mixing wheel having outer vanes at an outer extent thereof configured to mix the fluid in the vessel;
c. a dip tube extending downward through the upper lid, the dip tube terminating at a lower end in an input/output port covered with a filter, the input/output port and filter being located within 1-3 inches of the outer vanes of the mixing wheel; and
d. a pump system connected to an upper end of the dip tube for alternately causing perfusion outflow through the dip tube from the vessel or inflow through the dip tube into the vessel.

2. The bioreactor of claim 1, wherein the pump system is a bi-directional pump connected in line with the dip tube.

3. The bioreactor of claim 2, wherein the bi-directional pump is a peristaltic pump.

4. The bioreactor of claim 1, wherein the pump system includes two separate pumps each connected to separate lines leading to a branch point at an upper end of the dip tube, and a valve at the branch point connected to enable conversion of flow between the two lines and the dip tube.

5. The bioreactor of claim 1, wherein the filter is made of a porous material that is gamma-sterilizable.

6. The bioreactor of claim 5, wherein the filter is made of a material selected from the group consisting of sintered polyethylene mesh, polyvinylidene fluoride (PVDF) mesh, and stainless steel mesh.

7. The bioreactor of claim 1, wherein the filter is a cylindrical shape with an axis parallel to the horizontally-oriented axle of the mixing wheel.

8. The bioreactor of claim 7, wherein the dip tube includes an elongated vertical section that extends down from the upper lid that transitions at a gently curved 90° elbow section to a horizontally-oriented section terminating in the input/output port and filter.

9. The bioreactor of claim 1, wherein the input/output port and filter are located above the horizontally-oriented axle of the mixing wheel.

10. The bioreactor of claim 9, wherein the input/output port and filter are located at about a 45° angle extending from the horizontally-oriented axle above a horizontal plane through the horizontally-oriented axle.

11. A perfusion bioreactor, comprising:
a. a single-use vessel having a top edge and sized to contain a volume of fluid or medium, a vertically-oriented mixing wheel positioned within the vessel and having a horizontally-oriented axle supported within the vessel about which the mixing wheel rotates, the mixing wheel having outer vanes at an outer extent thereof configured to mix the fluid in the vessel;
b. a bioreactor system having a frame that receives the single-use vessel, and an upper lid that fits onto the top edge of the vessel and has access ports for a plurality of supply conduits and measurement instruments housed mounted to and controlled by the bioreactor system;
c. a dip tube extending downward through the upper lid, the dip tube terminating at a lower end in an input/output port covered with a filter located adjacent to and parallel to the horizontally-oriented axle of the mixing wheel; and
d. a pump system connected to an upper end of the dip tube for alternately causing perfusion outflow through the dip tube from the vessel or inflow through the dip tube into the vessel.

12. The bioreactor of claim 11, wherein the pump system is a bi-directional pump connected in line with the dip tube.

13. The bioreactor of claim 12, wherein the bi-directional pump is a peristaltic pump.

14. The bioreactor of claim 11, wherein the pump system includes two separate pumps each connected to separate lines leading to a branch point at an upper end of the dip tube, and a valve at the branch point connected to enable conversion of flow between the two lines and the dip tube.

15. The bioreactor of claim 11, wherein the mixing wheel has at least one magnetic or ferromagnetic driven element mounted thereon, the bioreactor system further including a drive system positioned outside of the vessel in close proximity therewith having at least one drive element configured to exert either an attractive or repulsive magnetic force to the driven element and rotate the mixing wheel.

16. The bioreactor of claim 11, wherein the filter is made of a porous material that is gamma-sterilizable.

17. The bioreactor of claim 16, wherein the filter is made of a material selected from the group consisting of sintered polyethylene mesh, polyvinylidene fluoride (PVDF) mesh, and stainless steel mesh.

18. The bioreactor of claim 11, wherein the filter is a cylindrical shape with an axis parallel to the horizontally-oriented axle of the mixing wheel.

19. The bioreactor of claim 18, wherein the dip tube includes an elongated vertical section that extends down from the upper lid that transitions at a gently curved 90° elbow section to a horizontally-oriented section terminating in the input/output port and filter.

20. The bioreactor of claim 11, wherein the input/output port and filter are located above the horizontally-oriented axle of the mixing wheel.

21. The bioreactor of claim 20, wherein the input/output port and filter are located at about a 45° angle extending from the horizontally-oriented axle above a horizontal plane through the horizontally-oriented axle.
